(19) United States
(12) Patent Application Publication
(10) Pub. No.: US 2023/0082459 A1
(43) Pub. Date: Mar. 16, 2023

(54) DEVICE FOR LARGE SPECIMEN RETRIEVAL

(71) Applicant: Covidien LP, Mansfield, MA (US)

(72) Inventor: Olesea Diaz-Chiosa, Naugatuck, CT (US)

(21) Appl. No.: 17/751,800

(22) Filed: May 24, 2022

Related U.S. Application Data
(60) Provisional application No. 63/244,301, filed on Sep. 15, 2021.

(51) Int. Cl. A61B 17/32

(52) U.S. Cl. CPC A61B 17/32002; A61B 2017/320024; A61B 2017/00287

(57) ABSTRACT

A device for removal of a tissue specimen from a patient includes a housing and a morcellator segment. The device includes a spindle capable of traveling through a longitudinal bore of the housing, and a specimen bag at a distal portion of the housing. In use, the specimen bag is extended from the distal portion of the housing and the spindle passes through the housing and specimen bag, exterior of the specimen bag, thereby forming an opening in the specimen bag. The spindle passes through a tissue specimen, at which point the spindle is retracted to bring the tissue specimen through the specimen bag, and into the longitudinal bore of the housing. The tissue specimen passes through the morcellator segment of the device, whereby the tissue specimen is broken into smaller pieces for ease of removal from the patient's body and further examination.

### CROSS-REFERENCE TO RELATED APPLICATIONS

This application claims the benefit of the filing date of provisional U.S. Patent Application No. 63/244,301 filed on Sep. 15, 2021.

### BACKGROUND

The present disclosure relates to tissue morcellation and, more specifically, to tissue morcellators which can be used for partial or total removal of body tissue or organs from a body cavity.

In minimally invasive surgical procedures, operations are carried out within the body by using elongated instruments inserted through small entrance openings in the body. The initial opening in the body tissue to allow passage of instruments to the interior of the body may be a natural passageway of the body, or it can be created by a tissue-piercing instrument such as a trocar, or by a small incision into which a cannula is inserted.

Minimally invasive procedures may be used for partial or total removal of body tissue or organs from a body cavity, e.g., partial or total hysterectomy, myomectomy, nephrectomy, cholecystectomy, lobectomy, and other procedures including thoracic, abdominal, laparoscopic, and endoscopic procedures. During such procedures, it is common that a cyst, fibroid, myoma, tumor, or other affected tissue or organ needs to be removed via the access opening or through a cannula. Various types of entrapment devices have been disclosed to facilitate this procedure. In many procedures where cancerous tumors are removed, removal of the specimen in an enclosed environment, e.g., a specimen bag, is highly desirable to inhibit seeding of cancer cells (i.e., portions of cancer cells contacting healthy tissue).

Several minimally invasive surgical procedures require the bulk removal of body tissue or organs through a limited surgical opening. As such, the tissue needs to be morcellated within the body cavity into smaller pieces of tissue to facilitate removal.

Improved morcellators for bulk removal of tissue remain desirable.

### SUMMARY

In an aspect of the present disclosure, a device incudes a housing defining a longitudinal bore and including a morcellator segment. The device includes a specimen bag affixed to a distal portion of the housing, the specimen bag having an opening at a distal portion of the specimen bag permitting passage of a tissue specimen through the specimen bag into the distal portion of the housing. The device also includes a spindle having an elongate body, a distal portion and a proximal portion, the spindle movable in relation to the longitudinal bore of the housing from a non-actuated position in which the spindle is positioned in a non-deployed state within the longitudinal bore of the housing to an actuated position in which the distal portion of the spindle is positioned in a deployed state externally of the distal portion of the elongate body of the housing. Retraction of the spindle in the deployed state guides the tissue specimen through the specimen bag, through the longitudinal bore of the housing, and through the morcellator segment.

In aspects, the morcellator segment has an inner wall including at least one blade extending into the longitudinal bore. In some aspects, the morcellator segment of the housing includes multiple blades.

In aspects, the spindle is further capable of rotating about a longitudinal axis of the elongate body of the spindle.

In aspects, the spindle has fins at the distal portion of the spindle, the fins adapted to extend from the longitudinal axis of the elongate body of the spindle in the deployed state.

In some aspects, a proximal portion of the fins extending away from the longitudinal axis of the elongate body of the spindle possess teeth for engaging the tissue specimen.

Retraction of the spindle into the longitudinal bore of the housing guides the tissue specimen through the specimen bag, and through the morcellator segment of the housing.

In some aspects, the device has a button on the housing for extending the spindle and for retracting the spindle.

In other aspects, the device has a button on the housing to control rotation of the spindle.

In other aspects the device has a button on the housing to introduce a gas through the housing into the specimen bag or to apply a vacuum through the housing.

In some aspects, a device includes a housing defining a longitudinal bore and including a morcellator segment, the morcellator segment having an inner wall possessing at least one blade extending into the longitudinal bore. The device includes a specimen bag affixed to a distal portion of the housing, the specimen bag having an opening at a distal portion of the specimen bag permitting passage of a tissue specimen through the specimen bag into the distal portion of the housing. The device also includes a spindle having an elongate body, a distal portion and a proximal portion, the spindle movable in relation to the longitudinal bore of the housing from a non-actuated position in which the spindle is positioned in a non-deployed state within the longitudinal bore of the housing to an actuated position in which the spindle is positioned in a deployed state externally of the distal portion of the elongate body of the housing. Retraction of the spindle in the deployed state guides the tissue specimen through the specimen bag, through the longitudinal bore of the housing, and through the morcellator segment.

In other aspects, a device includes a housing defining a longitudinal bore and including a morcellator segment, the morcellator segment having an inner wall possessing multiple blades extending into the longitudinal bore. The device includes a specimen bag affixed to a distal portion of the housing, the specimen bag having an opening at a distal portion of the specimen bag permitting passage of a tissue specimen through the specimen bag into the distal portion of the housing. The device also includes a spindle having an elongate body, a distal portion a proximal portion, and fins at the distal portion of the spindle, the spindle movable in relation to the longitudinal bore of the housing from a non-actuated position in which the spindle is positioned in a non-deployed state within the longitudinal bore of the housing to an actuated position in which the fins extend from the longitudinal axis of the elongate body in a deployed state externally of the distal portion of the elongate body of the housing. Retraction of the spindle in the deployed state guides the tissue specimen through the specimen bag, through the longitudinal bore of the housing, and through the morcellator segment.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a device provided in accordance with the present disclosure;

FIG. 2 is a side, partial, cross-sectional view of a distal portion of the device of FIG. 1 illustrating a specimen bag and a spindle within the distal portion of the device of the present disclosure;

FIG. 3 is a side, partial, cross-sectional view of the distal portion of the device of FIG. 2 after deployment of the specimen bag and the spindle from the distal portion of the device, with the spindle within the specimen bag;

FIG. 4 is a side partial, cross-sectional view of the distal portion of the device of FIG. 3 after deployment of the specimen bag and the spindle from the distal portion of the device, with a distal portion of the spindle exterior to the specimen bag;

FIG. 5 is a top view of the spindle depicted with the device of FIG. 4, after deployment of the spindle from the distal portion of the device;

FIG. 6 is a side cross-sectional view of the spindle of FIG. 5 after deployment from the distal portion of the device;

FIG. 7 is a side cross-sectional view illustrating the spindle engaged with tissue to draw the tissue into the device of FIG. 1;

FIG. 8 is a is a side cross-sectional view of the device of FIG. 1, illustrating the rotation and retraction of the spindle to bring the tissue into engagement with at least one blade within a morcellator segment of the device; and

FIG. 9 is a side cross-sectional view of the device of FIG. 1 after use.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

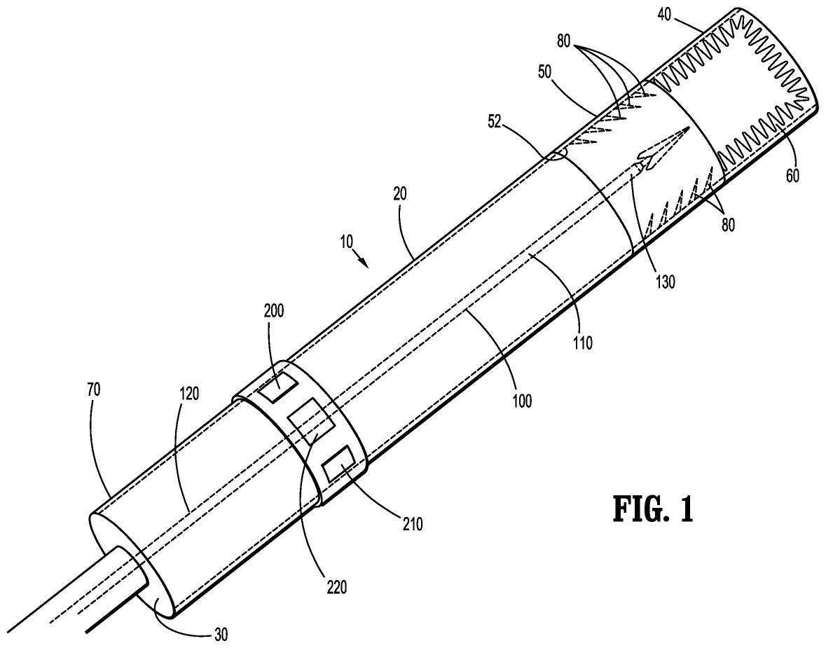

Referring now to FIG. 1, a device 10 is provided in accordance with the present disclosure. The device 10 includes a housing 20 that defines a longitudinal bore 30 therethrough. The housing 20 has a distal portion 40, a proximal portion 70, a morcellator segment 50, and a specimen bag 60 at the distal portion 40 of the housing 20.

The morcellator segment 50 possesses an inner wall 52 having at least one blade 80 extending into the longitudinal bore 30 to break up tissue. As shown in FIG. 1, multiple blades 80 may be present in the morcellator segment 50. Although not depicted, other morcellation mechanisms, including augers, may be used in the morcellator segment 50 instead of the blades 80.

The device 10 also includes a spindle 100 which passes through the longitudinal bore 30 of the housing 20. The spindle 100 has an elongate body 110, a proximal portion 120 and a distal portion 130. As described in greater detail below, in addition to being capable of moving distally and proximally within the longitudinal bore 30 of the housing 20, the spindle 100 is also capable of rotating about a longitudinal axis of its elongate body 110. The spindle 100 may be utilized to draw tissue into the morcellator segment 50 of the housing 20 as detailed below.

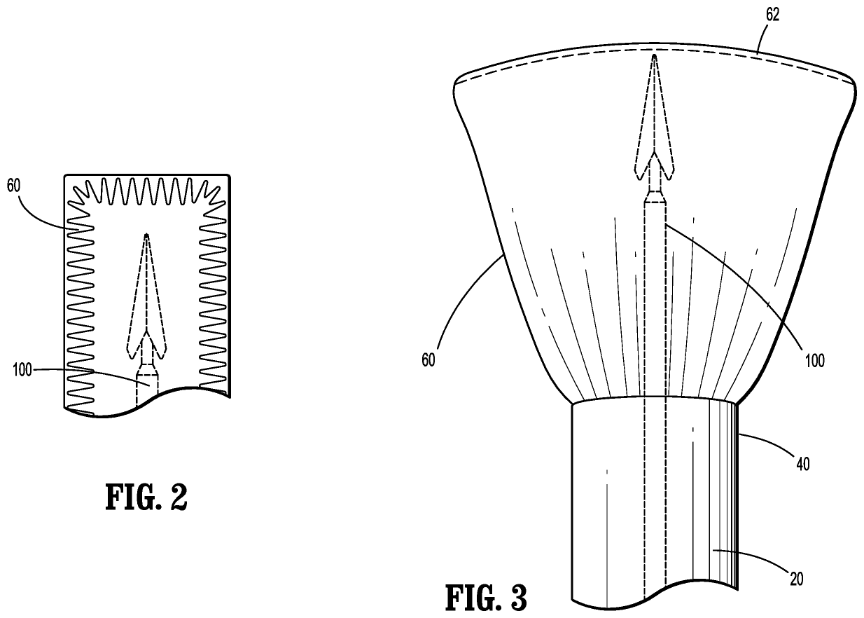

As noted above, the housing 20 includes a specimen bag 60 at the distal portion 40 of the housing 20. As depicted in FIGS. 1 and 2, prior to deployment, the specimen bag 60 is in a folded configuration within the distal portion 40 of the housing 20. The folded configuration permits the use of a specimen bag 60 having a larger volume (as compared to an unfolded configuration) when deployed from the device 10.

The housing 20 may be powered such that the housing 20 electromechanically extends, retracts, and/or rotates the elongate body 110 of the spindle 100, as well as expels gas or applies a vacuum to inflate or deflate the specimen bag 60. For example, as depicted in FIG. 1, the housing 20 can be connected to a power source (not shown) and include various control buttons 200, 210, 220 on a surface of the housing 20. These buttons may include a button 200 for extending and retracting the spindle 100 within the longitudinal bore 30 of the housing 20; a button 210 for rotating the spindle 100; and a button 220 for expelling or withdrawing air from the specimen bag 60. For examples of powered or manually operated devices, reference can be made to U.S. Patent Publication No. 2015/0141869 and U.S. Pat. No. 9,603,624, the entire disclosures of each of which are hereby incorporated by reference.

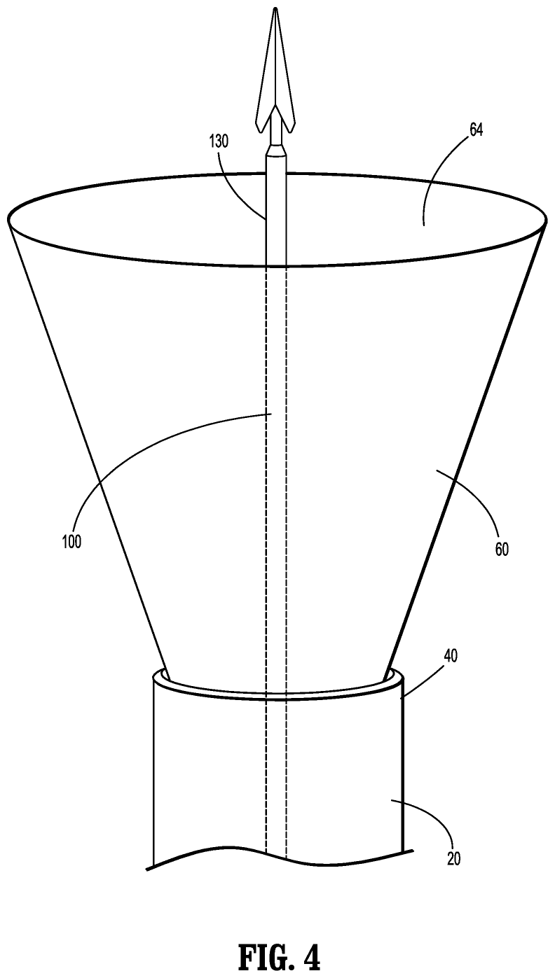

As depicted in FIG. 3, to deploy the specimen bag 60, air is introduced through the housing 20 (not shown) to inflate the specimen bag 60, thereby expelling the specimen bag 60 from the distal portion 40 of the housing 20. Once the specimen bag 60 has been deployed from the housing 20, the spindle 100 can move from a proximal position within the housing 20 to an extended position so that the distal portion 130 of the spindle 100 is within the specimen bag 60. Further extension of the spindle 100 causes the spindle 100 to perforate a distal portion 62 of the specimen bag 60, thereby forming an opening 64 (FIG. 4) of the specimen bag 60. The spindle 100 may then be further extended so that the distal portion 130 of the spindle 100 is exterior to the housing 20 and the specimen bag 60. As shown in FIG. 3, prior to deployment, the spindle 100 is capable of passing through tissue to be removed from a patient's body.

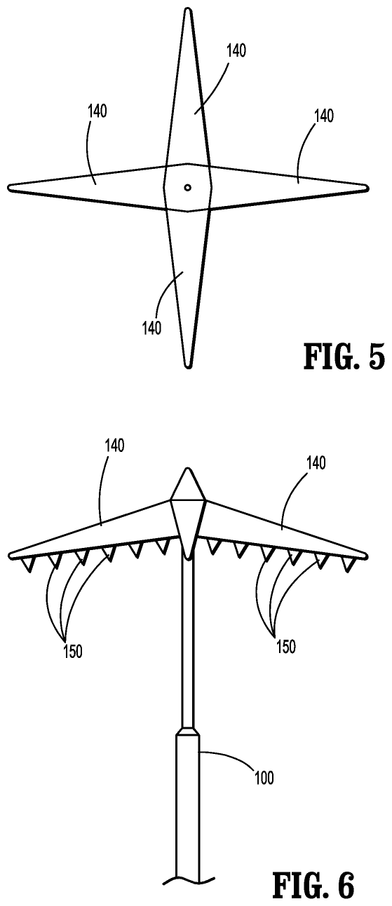

As depicted in FIGS. 5 and 6, the distal portion 130 of the spindle 100 has fins 140 which extend from the elongate body 110 in a deployed state, sometimes referred to herein as a deployed configuration. In aspects, the fins 140 may be extended manually to form the deployed state, for example by the use of graspers (not shown). The fins 140 have protrusions or teeth 150 on their proximal surface in the deployed state which engage with a tissue specimen to be removed (FIG. 6).

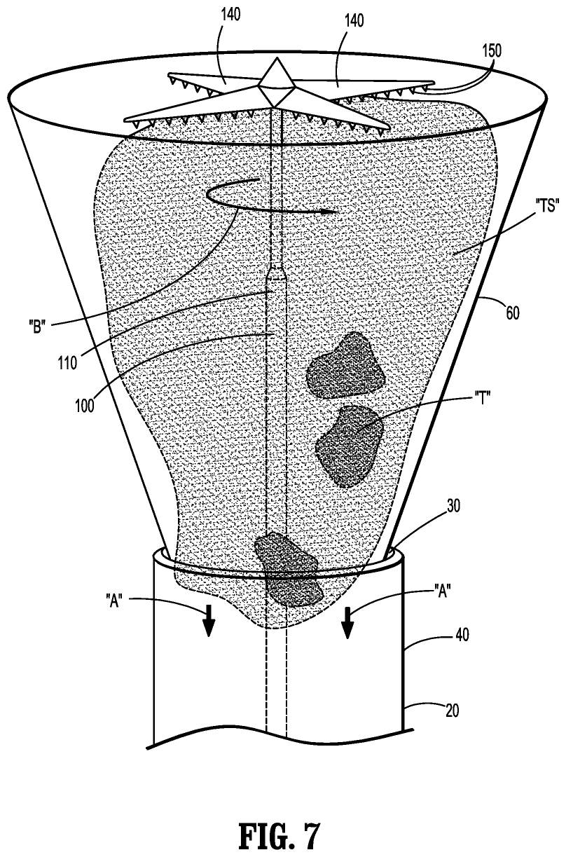

Use of the device 10 for morcellating tissue is disclosed in accordance with the present disclosure. Initially referring to FIG. 4, the spindle 100 is passed out of the housing 20 of the device 10 such that the distal portion 130 of the spindle 100 extends from the distal portion 62 of the specimen bag 60. The distal portion 130 of the spindle 100 is then brought into contact with a tissue specimen “TS” to be morcellated. The spindle 100 passes through the tissue specimen “TS” at which point the fins 140 at the distal portion 130 of the spindle 100 are extended in the deployed state, in aspects by the use of a graspers (not shown), so that the fins 140 extend from the elongate body 110 of the spindle 100. The spindle 100 is then proximally retracted, as indicated by arrows “A” in FIG. 7, so that the teeth 150 on the proximal portion of the fins 140 come into contact with the tissue specimen “TS”.

Continued retraction of the spindle 100 brings the tissue specimen “TS” through the specimen bag 60 and into the longitudinal bore 30 at the distal end 40 of the housing 20 of the device 10. The spindle 100 may be optionally rotated, indicated by arrow “B”, about the longitudinal axis of the elongate body 110 of the spindle 100. As depicted in FIG. 7, the tissue specimen “TS” may include tumors “T” within the tissue specimen “TS”.

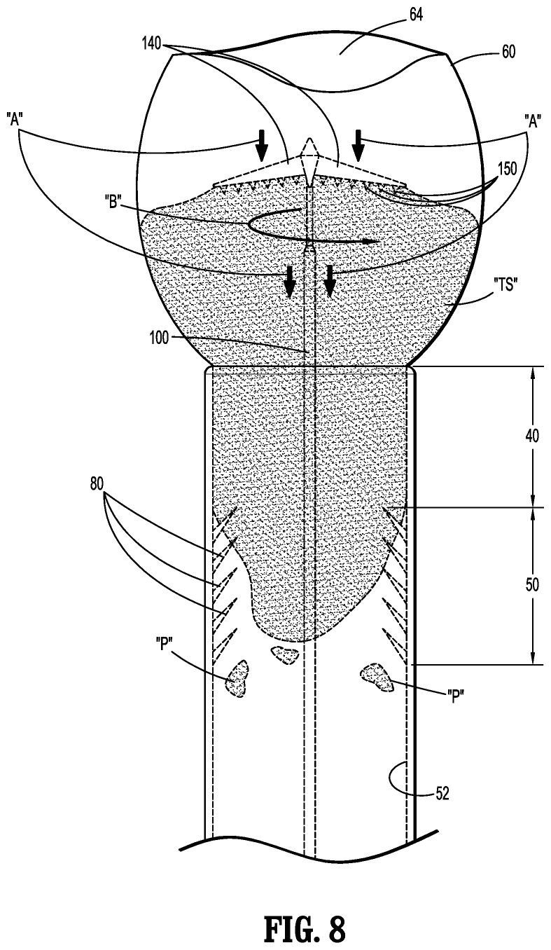

As depicted in FIG. 8, retraction of the spindle 100 carries the tissue specimen “TS” through the morcellator segment 50, where the tissue specimen “TS” contacts the blades 80. Rotation of the spindle 100 and proximal motion of the spindle 100 transports the tissue specimen “TS” through the morcellator segment 50 where the set of blades 80 break up the tissue specimen “TS” into smaller pieces “P” that may be collected and sent to pathology for examination.

Suction may be optionally applied to further assist in bringing the tissue specimen “TS” into the housing 20 of the device 10, including the morcellator segment 50.

As the tissue specimen “TS” passes through the specimen bag and into the housing 20 of the device 10, it enters into the morcellator segment 50 of the device 10 where it is broken up by the blades 80, thereby reducing the size of the tissue specimen “TS” and permitting collection of the tissue specimen “TS” entirely inside the device 10 which prevents seeding of the cancer cells within the patient's body.

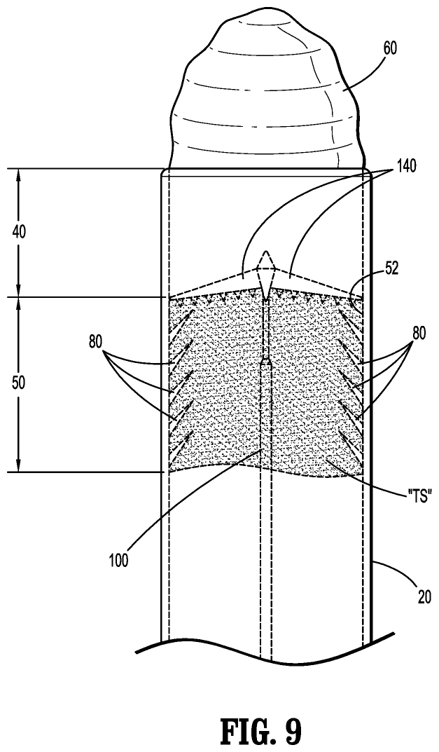

As depicted in FIG. 9, once the spindle 100 reaches its final, most proximal position in the device 10, a vacuum (not shown) may be applied to assist in closing the specimen bag 60 and withdraw the specimen bag 60 into the distal portion 40 of the housing 20 of the device 10. The device 10 can then be removed from the patient.

Devices in accordance with the present disclosure may be used in any soft-tissue surgery, that requires removal of large specimen tissue samples, including colorectal, liver resection, lung lobectomy and gynecological surgery. The devices can be adapted for both laparoscopic and robotic use, can be deployed through a natural orifice, or an umbilical incision having a size of less than 15 mm, and permit contactless specimen removal.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A device comprising:
a housing defining a longitudinal bore and including a morcellator segment;
a specimen bag affixed to a distal portion of the housing, the specimen bag having an opening at a distal portion of the specimen bag permitting passage of a tissue specimen through the specimen bag into the distal portion of the housing; and
a spindle having an elongate body, a distal portion and a proximal portion, the spindle movable in relation to the longitudinal bore of the housing from a non-actuated position in which the spindle is positioned in a non-deployed state within the longitudinal bore of the housing to an actuated position in which the distal portion of the spindle is positioned in a deployed state externally of the distal portion of the elongate body of the housing,
wherein retraction of the spindle in the deployed state guides the tissue specimen through the specimen bag, through the longitudinal bore of the housing, and through the morcellator segment.

2. The device according to claim 1, wherein the morcellator segment of the housing has an inner wall possessing at least one blade extending into the longitudinal bore.

3. The device according to claim 2, wherein the morcellator segment of the housing includes multiple blades.

4. The device according to claim 1, wherein the spindle rotates about a longitudinal axis of the elongate body of the spindle.

5. The device according to claim 1, wherein the spindle has fins at the distal portion of the spindle, the fins adapted to extend from the longitudinal axis of the elongate body of the spindle in the deployed state.

6. The device according to claim 5, wherein a proximal portion of the fins extending away from the longitudinal axis of the elongate body of the spindle possesses teeth for engaging the tissue specimen.

7. The device according to claim 1, wherein retraction of the spindle through the longitudinal bore of the housing guides the tissue specimen through the morcellator segment of the housing.

8. The device according to claim 1, further comprising a button on the housing for extending the spindle and for retracting the spindle.

9. The device according to claim 4, further comprising a button on the housing to control rotation of the spindle.

10. The device according to claim 1, further comprising a button on the housing to introduce a gas through the housing into the specimen bag or to apply a vacuum through the housing.

11. A device comprising:
a housing defining a longitudinal bore and including a morcellator segment, the morcellator segment having an inner wall possessing at least one blade extending into the longitudinal bore;
a specimen bag affixed to a distal portion of the housing, the specimen bag having an opening at a distal portion of the specimen bag permitting passage of a tissue specimen through the specimen bag into the distal portion of the housing; and
a spindle having an elongate body, a distal portion and a proximal portion, the spindle movable in relation to the longitudinal bore of the housing from a non-actuated position in which the spindle is positioned in a non-deployed state within the longitudinal bore of the housing to an actuated position in which the spindle is positioned in a deployed state externally of the distal portion of the elongate body of the housing,
wherein retraction of the spindle in the deployed state guides the tissue specimen through the specimen bag, through the longitudinal bore of the housing, and through the morcellator segment.

12. The device according to claim 11, wherein the morcellator segment of the housing includes multiple blades.

13. The device according to claim 11, wherein the spindle has fins at the distal portion of the spindle, the fins adapted to extend from the longitudinal axis of the elongate body of the spindle in the deployed state.

14. The device according to claim 13, wherein a proximal portion of the fins extending away from the longitudinal axis of the elongate body of the spindle possesses teeth for engaging the tissue specimen.

15. The device according to claim 11, further comprising a button on the housing for extending the spindle and for retracting the spindle.

16. The device according to claim 11, further comprising a button on the housing to control rotation of the spindle.

17. The device according to claim 11, further comprising a button on the housing to introduce a gas through the housing into the specimen bag or to apply a vacuum through the housing to the specimen bag.

18. A device comprising:
a housing defining a longitudinal bore and including a morcellator segment, the morcellator segment having an inner wall possessing multiple blades extending into the longitudinal bore;
a specimen bag affixed to a distal portion of the housing, the specimen bag having an opening at a distal portion of the specimen bag permitting passage of a tissue specimen through the specimen bag into the distal portion of the housing; and
a spindle having an elongate body, a distal portion a proximal portion, and fins at the distal portion of the spindle, the spindle movable in relation to the longitudinal bore of the housing from a non-actuated position in which the spindle is positioned in a non-deployed state within the longitudinal bore of the housing to an actuated position in which the fins extend from the longitudinal axis of the elongate body in a deployed state externally of the distal portion of the elongate body of the housing,
wherein retraction of the spindle in the deployed state guides the tissue specimen through the specimen bag, through the longitudinal bore of the housing, and through the morcellator segment.

19. The device according to claim 18, wherein a proximal portion of the fins extending away from the longitudinal axis of the elongate body of the spindle possess teeth for engaging the tissue specimen.
